(19) United States
(12) Patent Application Publication
(10) Pub. No.: US 2018/0011011 A1
(43) Pub. Date: Jan. 11, 2018

(54) SPECTROMETRIC IONIC IMPURITY MEASURING APPARATUS AND METHOD

(71) Applicant: UNIVERSITÉ LIBRE DE BRUXELLES, Bruxelles (BE)

(72) Inventor: Tomas FOLDES, Gosselies (BE)

(73) Assignee: Université Libre de Bruxelles, Bruxelles (BE)

(21) Appl. No.: 15/544,319

(22) PCT Filed: Jan. 8, 2016
(86) PCT No.: PCT/EP2016/050312

§ 371 Date: Jul. 18, 2017

(30) Foreign Application Priority Data
Jan. 20, 2015 (GB) 1500918.6

(51) Int. Cl. G01N 21/3577; G01N 33/18; G01N 21/3504

(52) U.S. Cl. CPC G01N 21/3577; G01N 21/3504; G01N 33/18

(57) ABSTRACT

A method for detecting and measuring the amount of an ionic impurity, notably formula (A) and/or formula (B) in a liquid sample, notably water, comprises: Introducing the liquid sample through a liquid inlet into a measurement cell, notably an optical cavity of an optical spectrometer; Causing vaporisation of the liquid sample by maintaining the pressure in the measurement cell below the saturated vapour pressure of the liquid sample; Causing the formation of gas-phase reaction product(s) of the ionic impurity; Measuring the amount of the gas-phase reaction product(s) of the ionic impurity in the measurement cell.

The present invention relates to an apparatus and a method for detecting and measuring ionic species, notably ionic impurities, in a liquid, particularly in water.

Control of water quality, notably monitoring of the presence and quantities of impurities (including contaminants, pollutants and disinfection by-products (DBPs)) in drinking water sources is of significant importance.

Ionic impurities contained in a liquid, for example present in river water, are generally measured by taking a sample and testing this in a laboratory situated away from the sampling location. Such laboratory testing involves delay, both for transportation and analysis of the sample, such a delay often being significantly longer that the time within which it is desirable to identify a change in the quantity of the ionic impurities in the water source.

One aim of the present invention is to provide an apparatus which provides fast or continuous measurements of one or more ionic impurities in a liquid sample with an appropriate level of sensitivity.

According to one of its aspects, the present invention provides an apparatus for detecting and measuring the amount of an ionic impurity in a liquid sample as defined in claim 1. Additional aspects are defined in other independent claims. The dependent claims define preferred and/or alternative embodiments.

The liquid sample may be water, notably water from a watercourse, groundwater, lake water, river water, well water, treated water, drinking water or waste water. The water sampled may comprise at least 95 wt % H₂O or at least 98 wt % H₂O. The pH of the water is preferably ≧6 or ≧6.5 and/or ≦9.2 or ≦8.5 or ≦8 or ≦7.5. Preferably, the amount of ionic impurity in the water or liquid sample is measured without adjustment of the pH of the sample.

The invention may be used:
to monitor the presence and/or concentration of a known or expected ionic species, for example to ensure that the concentration of the species remains within a pre-defined limit, notably for process control of an impurity present in a drinking water source for example to monitor the quantity of ammonium in a water source which is used as a drinking water source with or without further treatment; and/or
to monitor the presence and/or concentration of a known or expected ionic species introduced as a contaminant into a drinking water source, for example to detect a pesticide contamination from run off in a water source or to detect a deliberate attempt to poison a water source, for example with a cyanide compound;
to monitor the presence and/or concentration of a known or expected ionic species, for example a DBP, resulting from a water treatment, notably a water purification treatment for example the use of ozone as a disinfectant for the production of drinking water. One such example is for detection and/or monitoring of BrO⁻₃ which can be an undesired by-product from ozone purification of drinking water.

The temperature of the sample may be ≧0° C. or ≧4° C. and/or ≦25° C. or ≦20° C. At operating temperature, notably between 12° C. and 25° C., the liquid sample may have a saturated vapour pressure of ≦50 mbar, preferably ≦40 mbar, more preferably of ≦30 mbar. This allows vaporization of the liquid sample at low pressure.

The ionic impurity may comprise pollutants, contaminants and/or DBPs (Disinfection By-Products, for example resulting from reactions between organic and/or inorganic matter in water with chemical treatment agents during a water disinfection process). The ionic impurity may comprise: BrO₃⁻, NH₄⁺, CN⁻, HCOO⁻, CH₃COO⁻, IO₃⁻ and/or (CH₃)₂N₂⁺. The gas-phase reaction products may comprise HOBr, NH₃, HCN, HCOOH, CH₃COOH, HOI and/or (CH₃)₂NH.

The optical spectrometer may be based on cavity enhanced spectroscopy; it may be a cavity ring-down spectrometer, notably a continuous wave cavity ring-down spectrometer (cw-crds). This allows for rapid detection of small amounts of ionic impurities in a liquid sample. The apparatus may be used to detect a concentration of one or more impurities in the sample which is ≧0.1 ppt (part per trillion) or ≧1 ppb (part per billion) and/or ≦10 ppm (part per million) or ≦1 ppm. The delay between introduction of the sample and indication of the presence and/or quantity of the impurity may be ≦5 minutes, ≦2 minutes or ≦1 minute.

The light source may be a laser source; it may be an infrared light source, notably a near-infrared laser source or a near-infrared distributed feedback laser source. The light source may be configured to emit light at a wavelength which is ≧300 nm and/or ≦200000 nm, notably in the range 800 nm-1700 nm, preferably in the range 1200 nm-1700 nm, more preferably in the range 1400-1600 nm.

The wavelength(s) used for detecting and measuring the amount of the ionic impurity, notably the wavelength(s) of the light source, may be selected according to the ionic impurity to be detected. For example, the wavelengths may be selected within or over the range:
1470 nm to 1540 nm; notably from about 1527.03 nm to about 1527.06 nm and/or from about 1526.98 nm to about 1527.01 nm, particularly for detecting ammonia NH₃; and/or
1520 nm to 1550 nm, notably from about 1528.640 nm to about 1528.655 nm and/or from about 1537.40 nm to about 1537.45 nm, particularly for detecting hydrogen cyanide HCN; and/or
1410 nm to 1420 nm, preferably from 1411 nm to 1414 nm, notably from about 1412.92 nm to about 1412.98 nm, particularly for detecting HOBr; and/or
1422 nm to 1450 nm, preferably about 1438 nm, notably from about 1438.23 nm to about 1438.42 nm, particularly for detecting HCOOH; and/or
1420 nm to 1440 nm, notably from 1420 nm to 1435 nm, particularly for detecting CH₃COOH; and/or
1350 nm to 1450 nm, notably 1390 nm to 1410 nm, particularly for detecting HOI; and/or
1515 nm to 1540 nm, particularly for detecting (CH₃)₂NH.

Particularly when the ionic impurity to be detected and/or measured is NH₄⁺ the measurement may represent the combination of i) the ionic impurity (in this case NH₄⁺) and ii) corresponding dissolved compound(s), species or non-ionic form(s) (in this case ammonia NH₃) present in the liquid sample. One particular attribute of preferred embodiments of the present invention is the ability to detect and provide a measurement which comprises an impurity which is present in the liquid sample in the form of an ion, as opposed to an impurity which is present in the form of a dissolved gas or liquid. Thus, in this aspect, the present invention is distinct from prior art systems which are only capable of and/or only used to detect and/or measure a dissolved gas in the liquid sample, for example prior art systems used to measure the amount of ammonia gas NH₃ present in a water sample but not the amount of ammonium ions NH₄⁺ present. For example, at typical pH of drinking water, the concentration of impurity present in the form of dissolved gas (for example as ammonia NH₃) may be significantly lower than the concentration of the impurity present in the form of ions (for example as ammonium NH₄⁺). A further aspect of the invention relates to conversion of an ionic impurity originally present in the liquid sample into a non-ionic gas to facilitate detection of its presence and/or amount by spectroscopy.

The optical spectrometer may comprise an optical isolator, an adjustable focus free space coupler comprising a lens, notably an aspheric lens and/or an optical amplifier, notably a semiconductor optical amplifier.

The optical spectrometer may further comprise a measurement cell comprising an optical cavity comprised of at least two mirrors. The mirror(s) may be low-loss mirror(s); it may have a reflectivity at the wavelength of the light source of at least 98%, at least 99%, at least 99.5% or at least 99.9%.

The measurement cell may comprise an optical cavity; it may be provided by a solid envelope. The envelope may have the form of a tube, for example a glass, notably borosilicate glass tube. The length of the optical cavity may be varied, for example with a piezo element at one of its extremities, so that the measurement cell periodically passes through resonance, for example with the light beam.

The apparatus may comprise a vaporisation system adapted to vaporise the sample to be analysed. For example, the measurement cell may be connected to a vacuum system notably comprising a vacuum pump. In a preferred embodiment, the vacuum pump is configured to provide and/or to maintain low pressure inside the measurement cell, that is to say a pressure below atmospheric pressure, notably a pressure less than 1 bar, notably a pressure less than 100 mbar. Preferably, the measurement cell is maintained at a soft vacuum. The pressure inside the measurement cell may ≦50 mbar, ≦30 mbar, ≦20 mbar, ≦10 mbar and/or ≧10⁻³ mbar, ≧10⁻² mbar, ≧0.1 mbar or ≧1 mbar. The pressure inside the measurement cell is preferably less than the saturated vapour pressure of the liquid sample and/or at least a pressure wherein the mean free path is no more than 10 cm, preferably no more than 1 cm, more preferably no more than 1 mm. In a preferred embodiment, the mean free path is of about 10 μm.

The liquid inlet serves to allow controlled introduction of the liquid sample in to the measurement cell, notably the vacuum cell. The liquid inlet may comprise a valve and/or an orifice and/or a membrane filter.

The membrane filter may be a hydrophilic membrane filter and/or a porous filter, notably a porous filter having pores of at least 0.01 μm, at least 0.1 μm, at least 0.2 μm, and/or no more than 1 μm, no more than 0.5 μm. The membrane filter may be an unlaminated membrane filter; it may be a PTFE membrane filter or a polycarbonate filter. The membrane filter may allow a water flow between the liquid inlet and the measurement cell of at least 0.01 ml/min, at least 0.02 ml/min, at least 0.05 ml/min, at least 0.5 ml/min, at least 1 ml/min and/or no more than 10 ml/min, no more than 5 ml/min, no more than 1 ml/min, no more than 0.5 ml/min or no more than 0.1 ml/min through the membrane at a pressure difference of about 1 bar. The membrane may comprise hydrophilic materials, for example hydrophilic polycarbonate fibres, and/or comprise a hydrophilic coating. The membrane preferably allows passage of the ionic species with little or no hindrance so as to avoid altering its concentration in the liquid sample due to passage through the membrane; a hydrophilic membrane may be used to provide this effect.

The valve may be a solenoid-operated valve. The orifice of the valve and/or the orifice of the liquid inlet may have a size of less than 100 μm, preferably less than 80 μm and/or at least 40 μm, preferably at least 50 μm.

The apparatus may be used for on-line and/or off-line analysis.

An embodiment of the invention will now be described, by way of example only, with reference to the accompanying drawing of which:

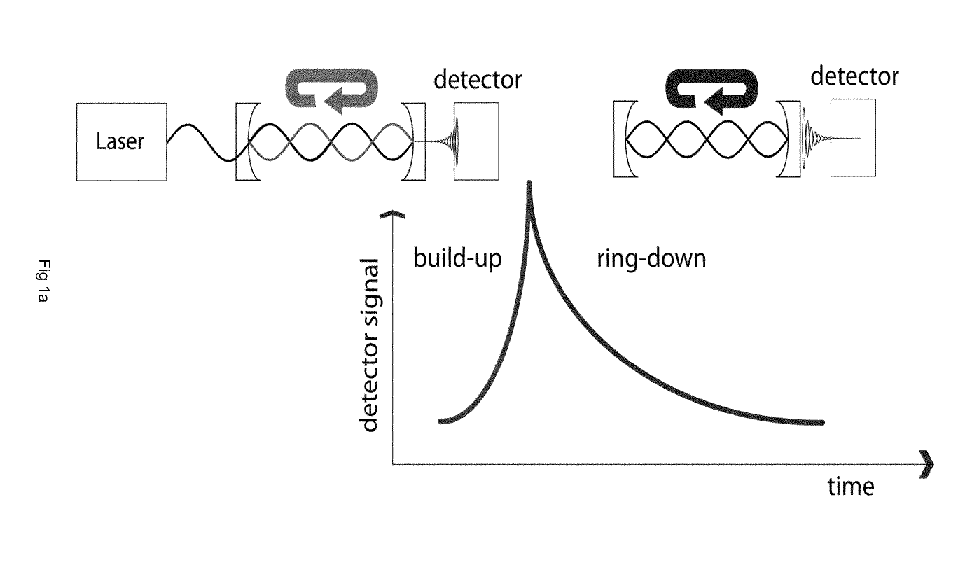

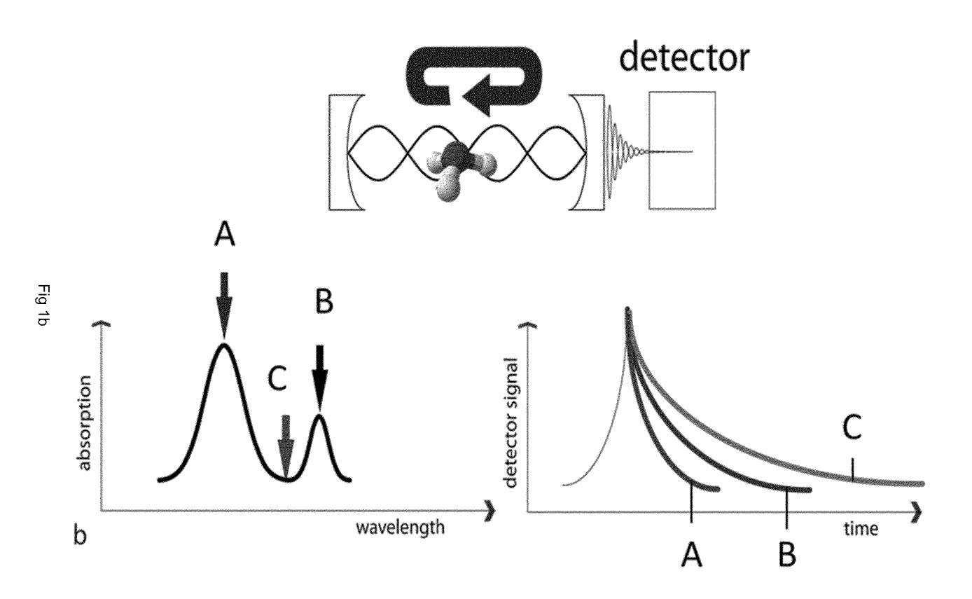

FIG. 1a and FIG. 1b show a schematic representation of the principle of operation of a continuous wave cavity ring-down spectrometer;

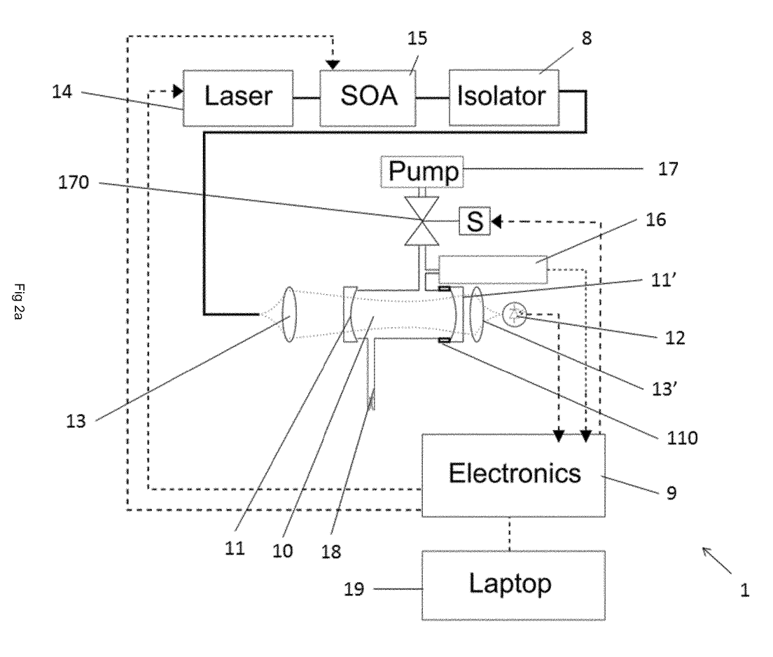

FIG. 2a and FIG. 2b show schematic views of alternative embodiments in accordance with the invention;

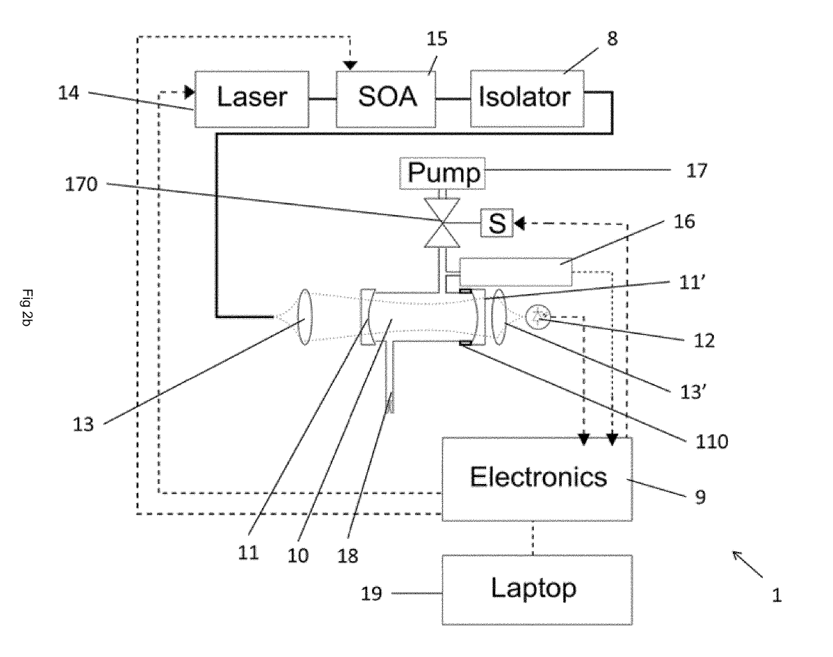

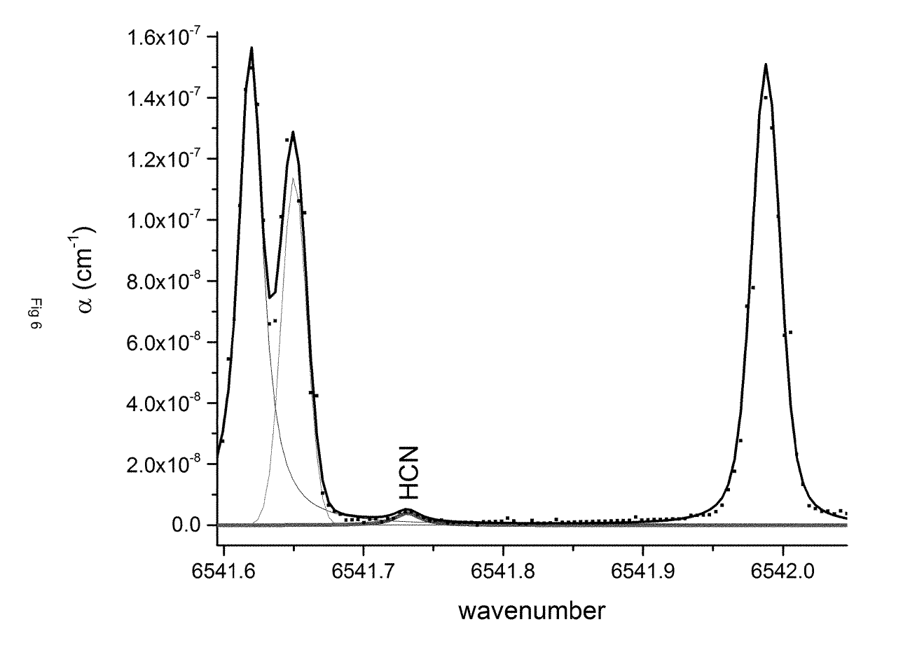

FIG. 3, and FIG. 6 are absorption spectra; and

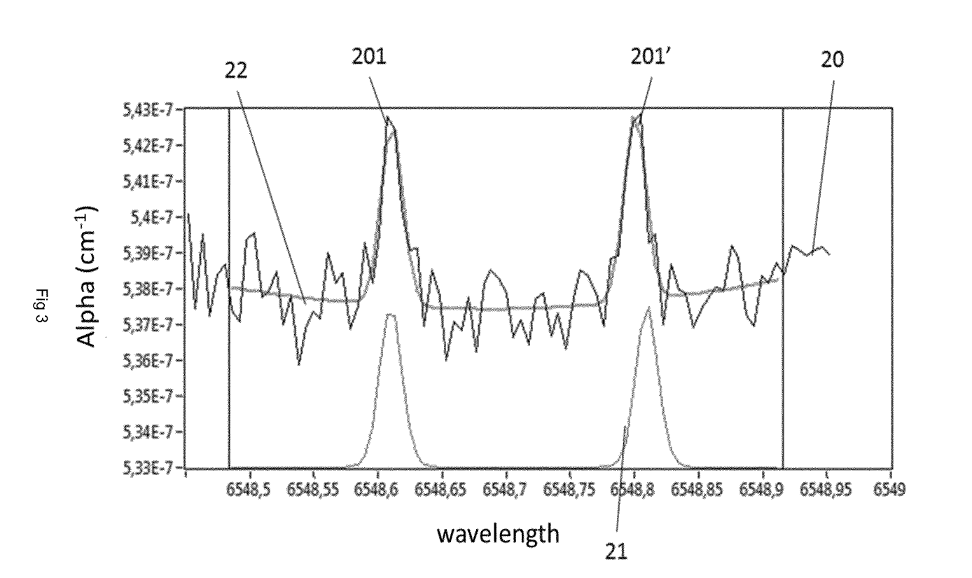

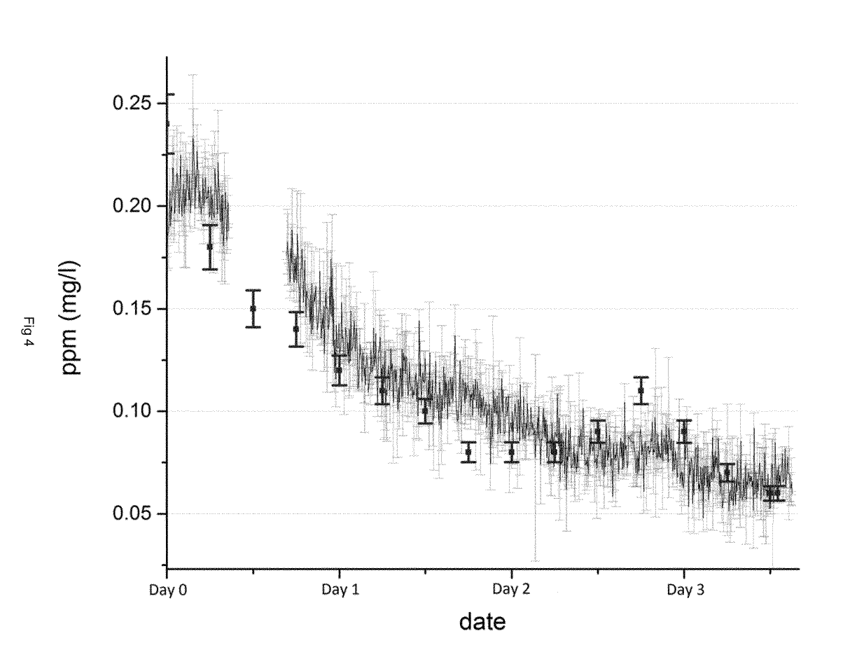

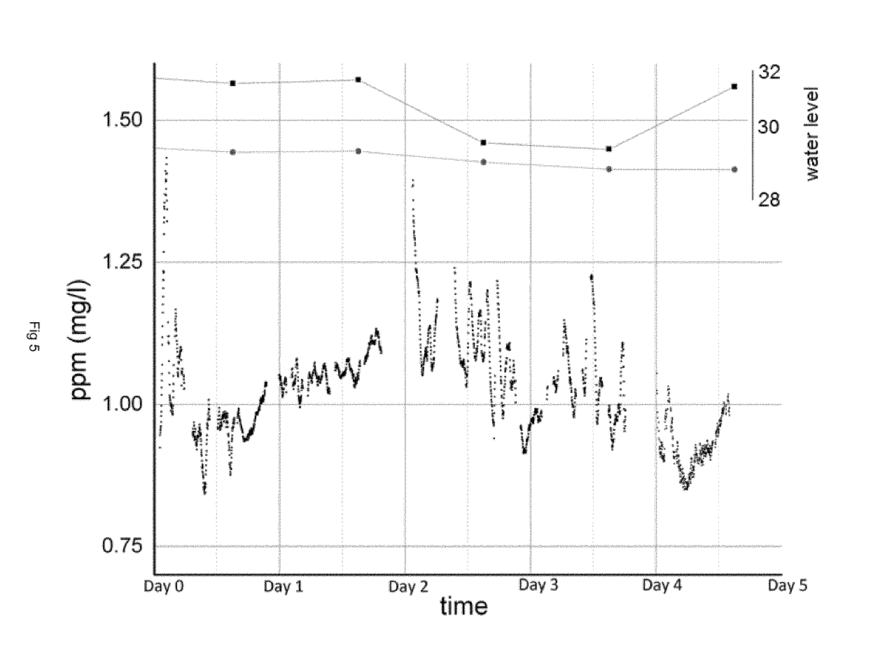

FIG. 4 and FIG. 5 show measured impurity concentrations over time.

The external dimensions of the apparatus 1 of FIG. 2a or FIG. 2b are 1200×290×460 mm. It consists of an optical cavity 10 (with mirrors 11, 11′ also functioning as the vacuum cell windows), a near-infrared DFB laser 14, a semiconductor optical amplifier 15, a liquid inlet 18 comprising a solenoid valve (as seen in FIG. 2a) or an orifice or a membrane filter (as seen in FIG. 2b), a vacuum pump 17, control and data acquisition electronics 9, and a control & analysis software running on a connected laptop computer 19. The power consumption is less than 80 W, running either on 230 V, which is converted by a laptop style external power supply to 12 V, or directly from a 12 V power supply. The instrument is not especially sensitive to vibration. Sample intake may be through ⅛ inch or smaller PFE tubing (not shown) connected to the inlet 18.

The principle of operation is depicted in FIG. 1a and FIG. 1b. The apparatus is based on continuous-wave cavity ring-down spectroscopy (cw-crds). Cw-crds instruments can achieve absolute measurements with high sensitivity and a good temporal resolution, thanks to many kilometres of active path length realized using a small (<1 m) closed-path optical cavity formed here by two mirrors. In this simple scheme, a tuneable narrow bandwidth continuous-wave (cw) laser is used to excite the length-modulated cavity. When resonance with the incoming laser beam is achieved, a rapid increase of the light intensity exiting the cavity can be detected. FIG. 1a illustrates the optical cavity 10 excited using a continuous wave laser. Then, once enough intracavity field has built up, the laser beam is interrupted using a fast switch to produce a ring-down event. The characteristic time of this exponential decay is called the “ring-down time”. The light intensity leaking out of the cavity is sampled and, based on its decay rate, the absolute absorption coefficient of the sample inside the optical cavity 10 is calculated at the set wavelength. By repeating this process at different wavelengths (by scanning the laser), the spectrum of the sample in the cavity is obtained as seen in FIG. 1b, wherein the ring-down time depends only on the absorption by the sample in the cavity (at the set wavelength), the mirror reflectivity, cavity length and the speed of light. FIG. 1b illustrates, on the left hand graph, an absorption curve and notably the absorption at wavelengths A, B and C whilst the right hand graph illustrates the respective ring-down decay for each absorption wavelength A, B and C. By scanning the laser over absorption lines of the sample, an absolute absorption spectrum of the sample is obtained. Because the decay rate of the ring-down signal is measured, and not the intensity, the technique is immune to laser power fluctuations.

The absorption coefficient α (in cm⁻¹) is calculated from the ring-down time τ (in seconds) via the formula:

$$α ( v ) = \frac{1}{c} ( \frac{1}{τ} - \frac{1}{{τ}_{0}} )$$

where c is the light velocity, τ₀ is the ring-down time of the evacuated cavity which depends on the residual transmittivity T of the low-loss mirrors 11,11′ and additional losses L that include the absorption by the dielectric coating and scattering of the surfaces and volumes. The reflectivity R can be calculated from the relation

R=1−T−L

Absorption features appear as lines superimposed on the spectrum baseline described by

$$\frac{1}{c {τ}_{0}}$$

A distributed feedback (DFB) laser module 14 is used as a laser source, which incorporates a fibre coupled semiconductor laser in a hermetically sealed package with a thermoelectric element, a 10 kΩ thermistor and a power monitoring photodiode. DFB lasers are commonly used in cw-CRDS setups operating in the near infrared range, because they are reliable, can be easily tuned (by temperature or current) and operate mode-hop free.

Preferably, a semiconductor optical amplifier (SOA) 15 is used as the high-speed optical shutter/switch although an acousto-optic modulator may be used. A semiconductor optical amplifier (SOA) 15 provides a power efficient solution for switching on and off the laser beam, and may provide additional amplification, for example up to about 100 mW optical power.

The laser beam further passes through an optical isolator 8 which protects the laser against optical feedback. An adjustable focus free space coupler with one f=7.5 mm aspheric lens 13 (Thorlabs CFC-8X-C) is used to couple and mode-match the exiting laser beam into the optical cavity 10 formed by two low-loss mirrors 11,11′. One of the mirrors 11′ is set on a kinematic mount 110 with integrated piezoelectric elements (Thorlabs KC1-T-PZ/M) allowing for length modulation of the cavity. The light exiting the cavity is focused by a second lens 13′ on a photodiode connected in a transimpedance circuit. The differential voltage signal from the circuit is used to trigger the data acquisition of the ring-down transients. The ring-down transients are sampled by a 2 MS/s data acquisition board (DAQ) (NI USB-6363). The digitalized signal is then transferred via USB connection to a notebook computer 19 and a fitting algorithm is used to fit the ring-down decays to obtain the spectrum. The spectrum is obtained from the variation of the ring-down time with the laser frequency.

The measurement is driven by a computer 19 via a USB connected data acquisition board (DAQ). 100 mA current is supplied to the DFB laser module 14 (laser) by a very low noise constant current source circuit (I). The laser is producing about 20 mW of optical power with 2 MHz bandwidth. The temperature of the laser and hence the wavelength is set using the thermo-electric controller board (TEC) the set point of which can be controlled via the DAQ. The laser beam exits the laser module 14 through a single mode optical fibre through a semiconductor optical amplifier (SOA) 15 and an optical isolator 8 via a mode-matching lens to the cavity. The SOA 15 acts as an amplifier (amplifying up to about 100 mW) and a fast optical switch. It is driven by 500 mA current that can be switched off very rapidly and is temperature stabilized to 25° C. (TEC). The length of the optical cavity 10 is modulated (by piezo elements 110 integrated into the optical mount) to periodically pass through resonance with the laser beam. The intensity leaking out of the cavity is monitored by a 3-stage transimpedance circuit around an InGaAs photodiode. The sample is introduced through the liquid inlet 18 into the cavity via PFE tubing (not shown) which, in the arrangement illustrated in FIG. 2a, is pressure regulated upstream by a small current-controlled proportional valve 180. The optical part of the instrument is isolated from vibration by wire rope isolators.

The apparatus was developed specifically for application on water and care was taken that surface materials minimize memory-effect problems with sticky molecules. The two low-loss mirrors 11,11′ (Layertec 106683) that form a 82 cm long optical cavity 10 also act as windows of the vacuum cell. The walls of the vacuum cell are formed by ¼ inch outside diameter (“OD”) borosilicate glass tubing (GPE scientific CG-713-01, precision ground OD tubing for use with PTFE ferrule Swagelok fittings). This ¼ inch glass tubing allows the use of standard ¼ inch Union Tee tube fittings (Swagelok PFA-420-3) in-line with the glass tubing as sample in/outlets. Flexible PFA tubing is connected perpendicularly via these tee tube fittings connecting the pump 17 and sample inlet 18.

The mirrors 11,11′ are housed in mirror holders inserted into kinematic mounts (Thorlabs KC1-T/M). The vacuum seal between the mirror holder and the mirrors 11,11′ and the mirror holder and the ¼ inch glass tubing is achieved via o-rings. The o-ring seal between the glass tubing and the mirror holders allows enough flexibility to align the cavity using the kinematic mounts without breaking the vacuum seal. The KC1-T/M kinematic mounts are compatible with the 30 mm cage system standard and 4 cage rods are used in addition to the post mounting to an optical construction rail (Thorlabs XE25) for additional stability. The 4 cage rods pass through the 2 kinematic mirror mounts, a cage plate that holds a lens (that focuses the exiting radiation from the cavity on the detector 12) and the printed circuit board (PCB) of the detector 12 and its housing. The detector housing is also post mounted to the rail.

The water sample is introduced into the cavity via suction by a small diaphragm pump (KNF N 84.4 ANDC). Pressure is measured by a (100 Torr full-range) baratron pressure gauge 16 with analogue voltage output read by the DAQ. Flow control is achieved by (i) a low-flow valve 180 upstream (illustrated in FIG. 2a) or (ii) an orifice 18 (or a membrane filter with a sufficiently low throughput) upstream and a valve 170 downstream (as illustrated in FIG. 2b). The valves are controlled by the DAQ as well.

During two measurement campaigns, an approach with a single low-flow valve 180 (Parker—Vso Low Flow—Normally Closed Proportional Valve, orifice size: 76 μm) upstream was used (illustrated in FIG. 2a). The valve was pulse-width-modulation (PWM) controlled by a PID Labview routine to maintain the pressure in the cavity at about 10 mbar (This was typically achieved with about 80% PWM duty cycle of the valve). The input tube diameter (the pneumatic connection on the valve is ⅛ inch OD manifold mount) was reduced in 2 steps to micro bore PTFE Tubing (Cole-Parmer EW-06417-11) to decrease the time necessary for the liquid sample to reach the cavity. The actual injected liquid flow rate is about 0.05 ml/min. This approach worked well during one test but it ran into problems during another test with water with high turbidity or carbonation because bubbles and dirt would clog the valve.

A second approach is to use a membrane filter, for example with a pore size of about 0.2 μm or about 0.01 μm upstream instead of the low-flow valve. Pressure can be regulated by choking the pumping rate by a solenoid valve 170 downstream, just before the pump (illustrated in FIG. 2b). The membrane filter is cut to size and fitted at one end of a union PFA fitting (Swagelok PFA-420-6) with the other end of the union fitted on the input flexible PFA tube. The tube with the fitting can be submerged in the sampled water. The throughput of this filter results in a pressure of about 5 mbar in the cavity if the pump is unchoked.

Similarly it would be possible to use a single laser drilled orifice (about 50 μm) instead of the membrane filter, but the orifice would probably require custom manufacturing and would be less immune to clogging.

The water injected containing dissolved species rapidly vaporizes and the produced gas-phase molecules and ions in the soft vacuum conditions undergo collisions with each other and the walls of the vacuum system. Products of the gas-phase reactions of these pollutants, contaminants and DBPs can be subsequently detected in the gas phase (e.g. NH₄⁺ can be detected as NH₃, CN⁻ can be detected as HCN and BrO₃⁻ can be detected as HOBr). The amount of these products is measured by cw-crds spectroscopy, the amount of water is determined from the total pressure or via cw-crds spectroscopy. The fraction of the two yields the parts-per concentration of the specific pollutant, contaminant or DBP.

Further species detected similarly could include formate (HC00⁻) detected as formic acid (HCOOH), acetate (CH₃COO⁻) detected as acetic acid (CH₃COOH) and iodate (IO₃⁻) detected as Hypoiodous acid (HOI), protonated dimethylamine (CH₃)₂NH₂⁺ as dimethylamine (CH₃)₂NH.

The water flow should be maintained in a range so that the pressure in the vacuum system stays below the saturated vapour pressure of water (so that all water entering the vacuum system can rapidly vaporize) and sufficiently high to have an adequate density of the species to be detected. (e.g.: the saturated vapour pressure of water at 12° C. is about 14 mbar, at 25° C. about 32 mbar).

Since the detection of the above mentioned species depends on collisions between the vaporized molecules, the technique may start to break down below about 10⁻³ mbar, as the mean free path of the molecules would be about 10 cm.

The mean free path of water molecules at the typical working pressure of about 10 mbar is about 10 μm.

The routine used to control the measurement of NH₃ includes a peak fitting algorithm to determine the area of Voigt profile peaks. By using a detailed understanding of the ammonia spectrum it is not always necessary to temperature stabilize the instrument, since the temperature dependence of the line intensity can be taken into account appropriately.

For the detection of ammonia, the water-vapour-induced pressure broadening coefficients of the 2 ammonia peaks 201, 201′ used for concentration measurements are determined. This allows determination of both the Gaussian (from temperature measurement) and Lorentzian (from pressure measurement) components of the fitted Voigt profiles, hence minimizing free parameters of the fit (only the baseline, area and position of the peaks are fitted). The position of the peaks is not fixed to allow compensation of the small drift of the laser current source and temperature controller.

The parts-per concentrations can be derived from the measured total pressure and temperature.

As seen in FIG. 3, a spectrum of a sample of tap water showed NH₃ peaks corresponding to 0.04 ppmv concentration. Tap water was sampled and injected into the vacuum cell. The scanning speed was set so that one scan would take less than a minute. The spectrum 20 was on-line fitted and concentration values could be displayed on a graph. Spectrum synthesized from initialization parameters is indicated at 21, and the final fit is indicated at 22.

The first test campaign was carried out at a “nitrifiltration” water treatment plant on the output of a newly replaced carbon filter. This campaign was triggered by a peculiar ammonium concentration measured during the first days of operation on the output of a previously replaced carbon filter, where very high ammonium concentration was detected (typically 1 sample per day is taken and analysed by standard analytical techniques). For this reason, when a new filter was installed, a frequent sampling procedure was planned (an automatic system that samples the water every 6 hours for analysis at an external laboratory was put in place) along with an on-line measurement by the apparatus described herein (also allowing validation). The measured concentrations during 3.5 days (along with the Continuous Flow Analysis (“CFA”) Automated Colorimetry measurements by the external laboratory on the samples taken) are shown in FIG. 4. The carbon filter operation was faultless in this case. The measurement campaign allowed the thorough validation of our measurement procedure. FIG. 4 shows concentration and the corresponding standard deviation measured by our instrument plotted in black and grey respectively. CFA Automated Colorimetry measurements by the external laboratory on samples taken every 6 hours are plotted in blue.

A second test measurement campaign was carried out at a slightly contaminated water catchment with unexplained ammonium concentration fluctuations. The measured concentrations are depicted in FIG. 5. Some data points had to be filtered out because the water was strongly carbonated and probably also contained solid matter particles, both of which can cause pressure fluctuations in the instrument. FIG. 5 shows total ammonium and ammonia concentrations measured during a 5 day period (lower trace). The catchment operated only during night hours, the day measurements corresponded to backwards flow in the pipes. The two traces at the top are water levels measured in the close-by piezometer wells.

Cyanide can be detected as HCN using the same principle as for ammonium and ammonia detection, as seen in FIG. 6 showing a concentration of 15 μg/I which is below the 50 μg/I limit set by EU regulations.

1-15. (canceled)

16. A method of detecting and measuring the amount of an ionic impurity in a liquid sample, the method comprising:
introducing the liquid sample through a liquid inlet of an optical cavity of an optical spectrometer;
causing vaporisation of the liquid sample by maintaining the pressure in the optical cavity below the saturated vapour pressure of the liquid sample;
causing the formation of gas-phase reaction product(s) of the ionic impurity;
measuring the amount of the gas-phase reaction product(s) of the ionic impurity in the optical cavity.

17. The method of claim 16, wherein the ionic impurity is selected from BrO₃⁻, NH₄⁺, CN⁻, HCOO⁻, CH₃COO⁻, IO₃⁻ and (CH₃)₂NH₂⁺.

18. The method of claim 17, wherein the ionic impurity is NH₄⁺ in water.

19. The method of claim 16, wherein the ionic impurity in the liquid sample comprises the ionic impurity in water.

20. The method of claim 16, wherein measuring the amount of the gas-phase reaction product(s) of the ionic impurity in the optical cavity comprises measuring the amount of the gas-phase reaction product(s) of the ionic impurity in the optical cavity using cavity ring-down spectrometry.

21. The method of claim 20, wherein the cavity ring-down spectrometry is continuous-wave cavity ring-down spectrometry.

22. The method of claim 16, wherein measuring the amount of the gas-phase reaction product(s) of the ionic impurity comprises introducing light from a light source into the optical cavity.

23. The method of claim 22, wherein the light has a wavelength in the range 800-5000 nm.

24. The method of claim 22, wherein the light is selected from: light from an infrared light source; and light from a near infrared distributed feedback laser source.

25. The method of claim 16, wherein causing the formation of gas-phase reaction product(s) of the ionic impurity comprises causing the formation of gas-phase reaction product(s) selected from HOBr, NH₃, HCN, HCOOH, CH₃COOH, HOI and (CH₃)₂NH.

26. The method of claim 16, wherein the pressure in the measurement cell during measurement of the amount of the gas-phase reaction product(s) is in the range 10⁻³ mbar to 50 mbar.

27. The method of claim 16, wherein the method comprises measuring a concentration of the ionic impurity in the liquid sample which is within the range of 0.01 ppt to 1 ppm.

28. A method of detecting and measuring a concentration of an ionic impurity in a water sample, in which the ionic impurity is present in the water sample in the range 0.01 ppt to ≦1 ppm, and in which the ionic impurity is selected from BrO₃⁻, NH₄⁺, CN⁻, HCOO⁻, CH₃COO⁻, IO₃⁻ and (CH₃)₂NH₂⁺, the method comprising:
introducing the water sample through a liquid inlet of an optical cavity of an continuous-wave cavity ring-down spectrometer;
causing vaporisation of the liquid sample by maintaining a pressure in the optical cavity in the range 20 mbar to 10⁻¹ mbar;
causing the formation of gas-phase reaction product(s) of the ionic impurity selected from HOBr , NH₃, HCN, HCOOH, CH₃COOH, HOI and (CH₃)₂NH;
measuring the amount of the gas-phase reaction product(s) of the ionic impurity in the optical cavity by continuous-wave cavity ring-down spectrometry.

29. The method of claim 28, in which the ionic impurity is NH₄⁺, and in which causing the formation of gas-phase reaction product(s) of the ionic impurity comprises causing formation of NH₃.

30. An apparatus for carrying out the method of claim 16, wherein the apparatus comprises
an optical spectrometer having an optical cavity, the optical cavity having a liquid inlet; and
a vacuum system comprising a vacuum pump, the vacuum pump being configured to provide a pressure of less than 50 mbar inside the optical cavity.

31. The apparatus of claim 30, wherein the optical spectrometer comprises a light source selected from a laser source, an infrared light source and a near infrared distributed feedback laser source.

32. The apparatus of claim 30 wherein the optical spectrometer is selected from a cavity ring-down spectrometer and a continuous-wave cavity ring-down spectrometer.

33. The apparatus of claim 30, wherein the optical cavity comprises at least two spaced mirrors having a reflectivity of at least 98%, each mirror being configured to reflect light through the optical cavity towards the other mirror.

34. The apparatus of claim 30, wherein the liquid inlet comprises a membrane filter.

35. The apparatus of claim 30, wherein the vacuum system is configured to provide a pressure in the measurement cell in the range 20 mbar to 10⁻¹ mbar.
